(19) United States
(12) Patent Application Publication
(10) Pub. No.: US 2014/0088650 A1
(43) Pub. Date: Mar. 27, 2014

(54) Polyaxial Connector for Spinal Fixation Systems

(71) Applicant: SPONTECH SPINE INTELLIGENCE GROUP AG, Stuttgart (DE)

(72) Inventors: Lino Taddia, Stockach (DE); Martin Pabst, Stuttgart (DE)

(73) Assignee: SPONTECH SPINE INTELLIGENCE GROUP AG, Stuttgart (DE)

(21) Appl. No.: 13/967,874

(22) Filed: Aug. 15, 2013

(30) Foreign Application Priority Data
Aug. 16, 2012 (DE) 102012016294.4

(51) Int. Cl. A61B 17/70

(52) U.S. Cl. CPC A61B 17/7032
USPC 606/267

(57) ABSTRACT

A polyaxial connector for spinal fixation systems has a lateral connector element having a first receptacle for a polyaxial connection to a spherical head of a pedicle screw and a second receptacle for a polyaxial connection to a spherical head of tulip that is configured to receive a connection rod clamped in the tulip by a locking element. A clamping piece is mounted in longitudinally-movable manner in the connector element between the receptacles. If the locking element is tightened, a pressure ram received in the tulip exerts a clamping force on the pedicle screw head, thereby fixing the connector element to the pedicle screw.

### CROSS-REFERENCE TO RELATED APPLICATIONS

This application claims priority of German patent application DE 10 2012 016 294.4 filed on Aug. 16, 2012. The full disclosure of this earlier application is incorporated herein by reference.

### BACKGROUND OF THE INVENTION

1. Field of the Invention

The present invention relates to a polyaxial connector for spinal fixation systems.

2. Description of Related Art

Spinal fixation systems are employed in the field of spinal surgery and serve for stabilising the spinal column, in particular in the case of traumas and degenerative diseases which require a correction, stabilisation or fixation of the spinal column. From the prior art, numerous spinal fixation systems are already known, these known mechanical systems usually comprising anchoring elements in the form of pedicle screws and connecting rods which connect the heads of the pedicle screws to one another. Known pedicle screws each have a shank, provided for bone anchoring, and a tulip-like receiving head connectable to a connecting rod. These “tulips” have a substantially U-shaped cross-section, the tulips being connected at their base directly to the shank of the pedicle screw, fixedly or by an arrangement of the ball-and-socket joint type (“polyaxial tulips”), and two free limbs which form a channel for receiving in each case one connecting rod and have an internal thread to enable screwing of a locking element against the connecting rod. In this way, the connecting rod can be connected to a pedicle screw by direct firm clamping.

Customary spinal fixation systems use in this regard rigid connecting rods fastened to the spinal column by the pedicle screws. Since the spinal column of each patient has a different anatomical structure, including different bone shapes and bone densities, the exact location of the pedicle screws and other parts of a spinal fixation system, including the correct shape of the connecting rods, are not established until during the operation, i.e. while the patient is already lying on the operating table. The reshaping of connecting rods, so that they are matched to the three-dimensional structure of the spinal column of each patient, is however very difficult, time-consuming and may lead to errors. Even if the three-dimensional anatomical orientation of pedicle screws is already determined before the operation, it is often necessary to re-orient these screws in the pedicles and/or readjust their penetration depth, since the force exerted for firmly clamping the connecting rod in the above-described systems acts directly on the pedicle screw. This quite often requires complete removal of a pedicle screw, particularly if the spinal deformation is not as expected or the space required for the pedicle screw is insufficient. However, removing, replacing or retightening pedicle screws jeopardises the fragile bone structure of the pedicles around the screw shank and requires additional operating time.

In order to eliminate the above-mentioned disadvantages, spinal fixation systems have already been developed which provide a lateral connector between the receptacle for the connecting rod, and the pedicle screw, such that the connecting rod can be fastened laterally offset with respect to the longitudinal orientation of the pedicle screw. The connection between the pedicle screw and the connecting rod can in this case take place after the insertion of the pedicle screw, so that no additional retightening or realignment of the pedicle screw is required. Possible loosening caused by this is therefore avoided.

A lateral connector for spinal fixation systems of this kind is known from US 2003/0045879 A1, which connector allows both rotation about the longitudinal axis of the pedicle screw with the possibility of substantial tilting from the rotational plane by a polyaxial connection, and adjustment of the lateral distance between the pedicle screw and the connecting rod with simultaneous possibility of rotation about the longitudinal axis of a rod-shaped extension of the connector pointing away from the pedicle screw head. In this case, a tulip-shaped receptacle for the connecting rod is displaceably and rotatably mounted on the rod-shaped extension of the connector and is fixable by the locking element for the connecting rod, which element is situated in the tulip-shaped receptacle. The rotational and tilting movement is achieved by a second part of the connector of the ball-and-socket joint type, at the lower side of which there is arranged a bearing which can receive a spherically symmetrical pedicle screw head, and at the upper side of which there exists a second locking or clamping element for fixing.

Since the connecting rod can be rotated and displaced in the receptacle before being fixed, 7 degrees of freedom of movement therefore result for this connector in the unfixed state.

US 2007/0288004 A1 discloses a lateral connector for spinal fixation systems having a head part which can be mounted on a spherically symmetrical screw head of a pedicle screw, resulting in a connection of the ball-and-socket joint type with three degrees of freedom of rotation. This connection can be polyaxially fixed by a locking element. The head part is connected to an arm, on which a tulip-like receptacle for the connecting rod is rotatably mounted, to be precise in such a way that an additional rotation in the plane in which the arm lies is possible. To fix the connecting rod inside the tulip-like receptacle, a second locking element is required.

With the possibilities of rotation and displacement of the connecting rod in the receptacle, 6 degrees of freedom of movement in the non-fixed state thus result.

A further lateral connector known from US 2007/0238335 A1 has no direct polyaxial connection to the pedicle screw, in contrast to the connectors shown hitherto. The pedicle screw has no spherical head but instead a thread in order to be able to firmly screw on a connecting part, provided with a fastening hole, of the connector by means of a fastening nut after this connecting part has been slipped on. Before the fastening, a possibility of rotation of the connector about the longitudinal axis of the pedicle screw results therefrom, the fastening height of the connecting part being predetermined by an annular shoulder which is situated on the screw shank and the outside diameter of which is greater than the inside diameter of the fastening hole. The connecting part, fastened in this way, of the connector is firmly connected, via a rotatable coupling, to a clamping device for the connecting rod, enabling a rotation about a transverse axis lying transversely with respect to the longitudinal orientation of the pedicle screw and running through the longitudinal axis of the lateral connector. The clamping device includes a locking element for the connecting rod, which element, by means of a holding clamp arranged displaceably in the clamping device and of interlocking toothing arrangements of a coupling part between the connecting part and the clamping device, serves at the same time for blocking the rotational movement about the longitudinal axis of the lateral connector.

In view of the fact that the connecting rod can be displaced and rotated before being fixed in the clamping device, this connector therefore allows 4 degrees of freedom of movement in the non-fixed state.

The aforementioned lateral connectors possess 4, 6 or 7 degrees of freedom of movement in the non-fixed state and each require two locking elements for final fixing. Each locking element which is to be adjusted by a surgeon during an operation complicates, however, not only a fixation system as such, but also additionally increases the adjusting and operating time.

In order to remedy this, there have already been developed spinal fixation systems having lateral connectors which, despite the plurality of degrees of freedom of movement in the non-fixed state, make do with only one locking element in order to be changed into the fixed state.

Such a lateral connector with 5 degrees of freedom of movement in the non-fixed state and only one locking element is known from US 2007/0156142 A1.

This connector is similar in design to the last-mentioned transverse connector having four degrees of freedom of movement in the non-fixed state, with the essential difference that the connection between the pedicle screw and the connector is embodied by a different kind of clamping device which encloses in the manner of a ring a cylindrical pedicle screw head without a thread and is attachable thereto such that it can slide and be fastened, thus providing, besides the rotational movement about the longitudinal axis of the pedicle screw, additionally also a height adjustability in the direction thereof. The fixing of this clamping device to the pedicle screw takes place via a coupling element, formed thereon and additionally displaceable in the longitudinal direction, simultaneously with the tightening of the locking element for the connecting rod in the other clamping device, so that changing into the fixed state is possible with only one locking operation, i.e. in one step.

Further lateral connectors of this kind may also be found in DE 10 2004 056 091 B4. These connectors consist in principle of two clamping devices which are connected to one another via a cardan-joint-like and additionally longitudinally displaceable coupling piece. While the first clamping device serves for connection to a connecting rod of a spinal fixation system, the second clamping device serves for connection to a pedicle screw head. If the second connecting device as proposed is configured for receiving and connection to a spherical-head-like pedicle screw head, this results in a maximum number of 8 degrees of freedom of movement in the unfixed state, if the possibilities of rotation and displacement of the connecting rod in the first clamping device before fixing are included. In order to fix all the free movement possibilities in one step, it is sufficient to tighten a tensioning member in the coupling piece.

The lateral connector shown here is admittedly extremely flexible owing to its 8 degrees of freedom of movement and meets the condition of adjustability in one step, but owing to the large number of its individual parts it is of quite complicated design and because of its grooved coupling parts is prone to soiling and difficult to clean.

An improvement in this regard is constituted by the lateral connector known from WO 2010/108655 A2.

This connector consists essentially of an elongate, so-called head element which is mounted rotatably and height-adjustably about a cylindrical pedicle screw head, so that the head element can move in its elongate extent in different planes extending at right angles to the longitudinal axis of the pedicle screw, and of a polyaxially movable tulip as receiving and connecting part for a connecting rod. The polyaxial tulip is attached to a part of the head element situated at a distance from the pedicle screw and points with its u-shaped receiving part away from the shank of the pedicle screw. Via a clamping element in the form of a tensioning screw situated in the polyaxial tulip, it is possible to fix simultaneously not only the connecting rod but also the tulip itself and the rotatable and height-adjustable connection of the connector to the pedicle screw head.

This takes place via pins which are located at right angles to one another and have oblique bearing surfaces and a one-way coupling which are accommodated in bores inside the head element.

This known construction of a lateral connector not only opens up 7 degrees of freedom of movement in the non-fixed state owing to its numerous polyaxial rotation possibilities and the possible height adjustability, but also allows fastening of a connecting rod running directly or partially above the pedicle screw head itself.

However, the disadvantage of this known, already extremely flexible structure is that a special tool to be inserted into the head element is required to release the one-way coupling.

### SUMMARY OF THE INVENTION

The object of the present invention is to eliminate these deficiencies, while at the same time retaining maximum flexibility and furthermore enabling fixing in one step.

In an embodiment, this object is achieved by a polyaxial connector for spinal fixation systems having a lateral connector element having two receptacles for polyaxial connecting arrangements. The first receptacle, which is situated at one end of the connector element and is accessible from the upper and lower side of the latter, serves for connection to a spherical pedicle screw head and is formed substantially as a ball-and-socket joint bearing for receiving the pedicle screw head which is insertable from the lower side of the connector element. The second receptacle, which is situated at the other end of the connector element, integrates a polyaxially movable tulip which projects to the upper side of the connector element and the upper part of which is provided, in a known manner, with a locking element for connection to a connecting rod of a spinal fixation system and for its own locking and the lower part of which, received in the connector element, has a spherical head having a pressure ram movably mounted therein and acted upon by a spring. The connector further comprises a clamping piece which is mounted in a rotationally and longitudinally-movable manner in the connector element between the receptacles and via which, with the aid of the locking element and the pressure ram, when fixing a connecting rod in a slot of the tulip, simultaneously a clamping force is exerted on the pedicle screw head situated in the first receptacle and via which an engagement of the pedicle screw head in the connector element is made possible by means of the spring.

The advantages achieved by the invention lie especially in the high flexibility in spite of the relatively small number of components, with 8 degrees of freedom of movement being available in the non-fixed state, in a simple and quick change from a movable into a completely fixed state and vice versa and in the full incorporation of the necessary clamping and unlocking mechanism inside the connector element. Owing to the high number of geometrical freedoms, in particular the increase of the polyaxial connection possibilities, it is possible to orient a connecting rod in any desired position within a limited distance relative to the vertebral bodies and subsequently fix it with a good force fit. The fixing of the position of the vertebral bodies with respect to one another can be chosen largely independently of the geometry of the connecting rod. It is therefore possible to fix the vertebral bodies in the position desired by the surgeon, without this position being influenced by the geometrical shape of the posterior rods. The connector is thus designed such that, in the non-fixed state and during the fixing operation, no forces or moments are built up between pedicle screw and connecting rod within this system. Owing to the construction provided, the initial transmission of forces to the pedicle screw/pedicle interface is substantially reduced. This reduction of the load has the result that consequential effects such as loosening of pedicle screws and screw fractures occur less frequently.

Further advantageous configurations or developments of the polyaxial connector are found in Subclaims 2 to 8, and uses of the claimed polyaxial connectors in the spinal fixation systems respectively claimed by claims 9 and 10.

### BRIEF DESCRIPTION OF THE DRAWINGS

Embodiments of the invention which reveal further advantages and special features are illustrated in the drawings and described in more detail below. In the drawings

FIG. 1: shows a perspective view of a spinal fixation system (5) according to the invention implanted in a section of the human spinal column;

FIG. 2: shows the spinal fixation system (5) according to the invention illustrated in FIG. 1, in plan view;

FIG. 3: shows the spinal fixation system (5) according to the invention illustrated in FIG. 1, in side view;

FIG. 4: shows a connector (2) according to the invention with engaged pedicle screw (1), in section;

FIG. 5: shows a connector (2) according to the invention with engaged pedicle screw (1), in projection;

FIG. 6: shows a clamping piece (28) in perspective view;

FIG. 7: shows a connector (2) according to the invention in horizontal partial section;

FIGS. 8,9: show the degrees of freedom of movement of the polyaxial connector (2) in the non-fixed state.

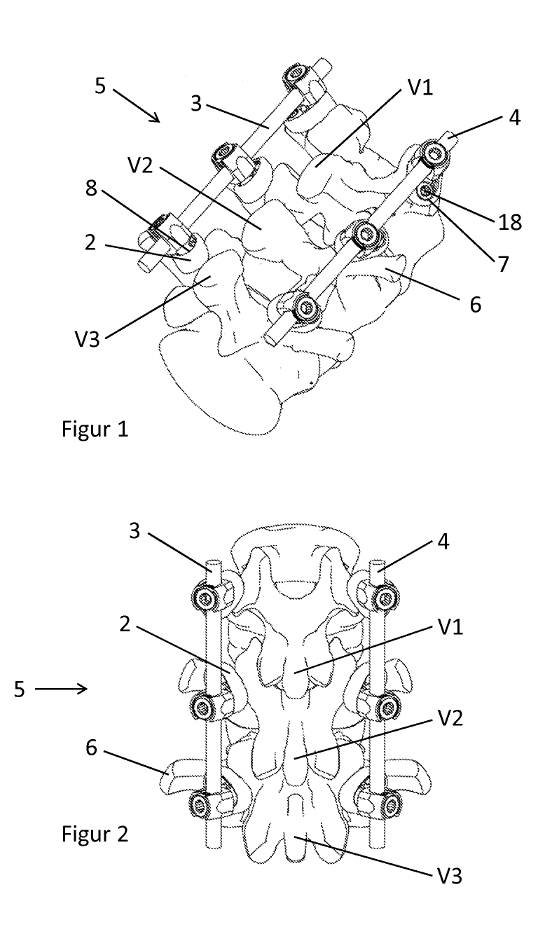

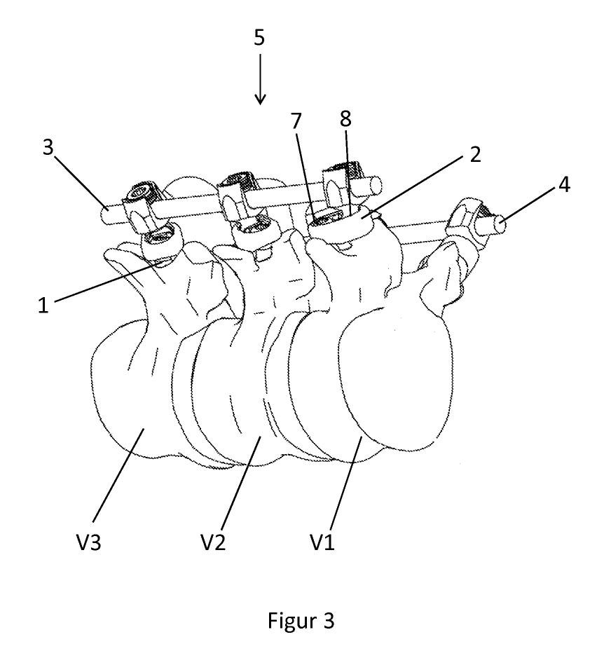

FIGS. 1 to 3 each illustrate, by way of example, a spinal fixation system (5) according to the invention implanted in a section of the human spinal column, in perspective view in FIG. 1, in plan view in FIG. 2, and in side view in FIG. 3. The section comprises in each case three successive vertebrae (V1, V2, V3), the adjacent vertebrae not being shown in FIGS. 1 to 3 for the sake of simplicity.

The spinal fixation system (5) comprises two connecting rods (3,4) extending on both sides of the spinal column in the longitudinal direction of the spinal column. In the example shown, the connecting rods (3,4) are illustrated as straight, flexurally rigid bars which can be made of a suitable material, e.g. titanium. In other embodiments, these connecting rods (3,4) may be curved or otherwise pre-bent. The connecting rods (3,4) may have a round, oval or polygonal cross-section.

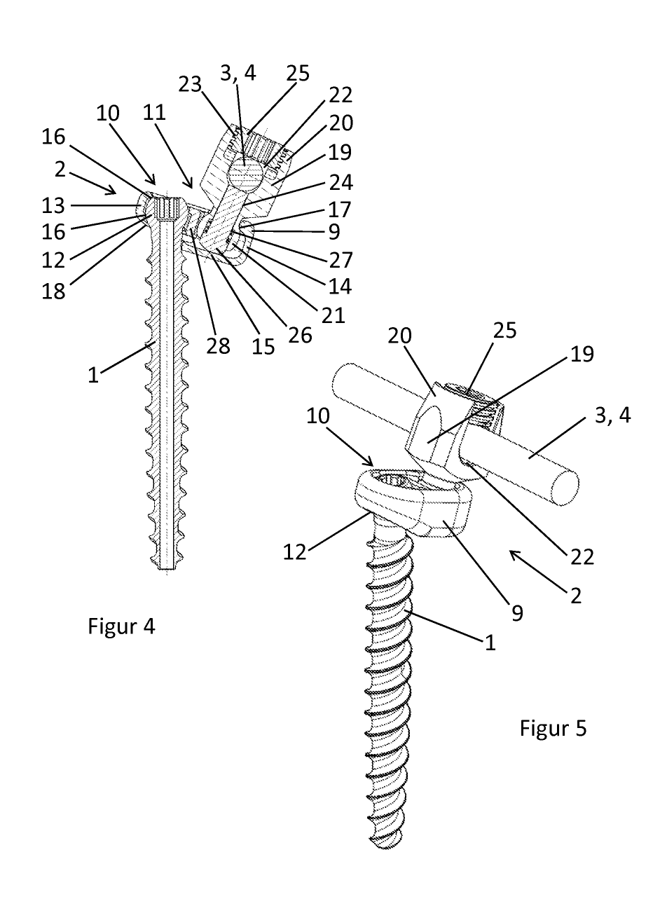

The connecting rods (3,4) are fastened to the vertebrae (V1,V2,V3) with the aid of pedicle screws (1), which are screwed into the respective pedicles (6) of the vertebrae (V1,V2,V3), and polyaxial connectors (2), which connect the pedicle screws (1) to the connecting rods (3,4). As will be apparent from the following description of the polyaxial connectors (2) according to the invention and with reference to FIGS. 4 to 9, these polyaxial connectors (2) each have two interfaces in the form of polyaxial connecting arrangements (7,8). The number of eight degrees of freedom of movement resulting therefrom enables multifarious orientations between a connecting rod (3,4) and a pedicle screw (1). Thus, for example, the straight, flexurally rigid connecting rods (3,4) can be connected to the pedicle screws (1) even though the latter are oriented neither parallel to one another nor in a line, as can best be seen from FIG. 2. Furthermore, the connecting rods (3,4) can run directly above the pedicle screw heads (18) or transversely with respect to them.

FIG. 4 shows in section the polyaxial connector (2) according to the invention when already connected to a pedicle screw (1), consisting of a connector element (9), a tulip (19) with tulip head (20) and a spherical head (21). In the tulip head (20) there is a slot (22) for receiving a connecting rod (3,4). Also formed in the tulip head (20) is a thread (23) for screwing in a locking element (25) having an external thread. At the lower end, the tulip head (20) merges into the spherical head (21). The latter has a central, stepped bore (24) for axially guiding a spring-loaded pressure ram (26). The spring (27) acts as a compression spring, which is supported on the one hand in the stepped bore (24) and on the other hand on the collar of the pressure ram (26).

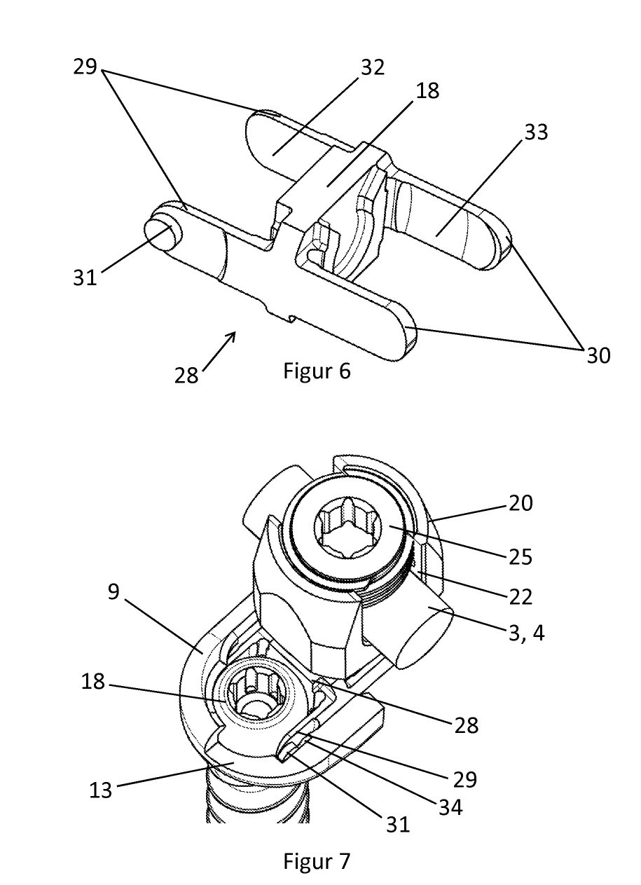

As can be seen from FIG. 5, all the outer corners and edges of the connector element (9) are rounded in order to improve the handling and reduce the risk of injury. It can also be seen in this view that the connector element (9) has a section (12) which extends, in the region of the first receptacle (10), approximately from the centre towards the side obliquely upwards, in order to enable a greater pivoting angle of the pedicle screw (1) and easier insertion of the pedicle screw head.

As further shown in FIG. 4, the left-hand housing wall (13) of the connector element (9) is of U-shaped form for bearing the pedicle screw head (18). The opposite housing wall (14) of the connector element (9) is likewise of substantially U-shaped design and merges into the housing bottom (15). At the upper border, the housing wall (14) has an inner bead or a semi-annular enlargement as a clamping surface (17).

Situated between spherical head (21) and pedicle screw head (18) is a clamping piece (28), which in plan view has a H-shaped basic structure with straps (29) and (30), as illustrated in FIG. 6. The parallel straps (29) are cylindrically shaped at their opposite, inner surfaces (32) for bearing on the pedicle screw head (18), so that the pedicle screw head (18) comes into line contact when it engages between the straps (29). To facilitate the engagement, the straps (29) are resilient. The outer surfaces of the straps (29) have cylindrical pins (31) formed integrally diametrically on a geometrical axis.

The straps (30) opposite the straps (29) have, on the inner surfaces (33), likewise a cylindrical shape which can be brought into line contact with the spherical head (21). The spherical head (21) also engages, during which the straps (29) help to keep the clamping piece (28) in the correct position until the pedicle screw head (18) is engaged, for which purpose these straps (30) likewise have resilient properties. The cylindrical surfaces (33) merge, at the outer end of the respective straps (30), into a spherical shape, adapted to the spherical head (21).

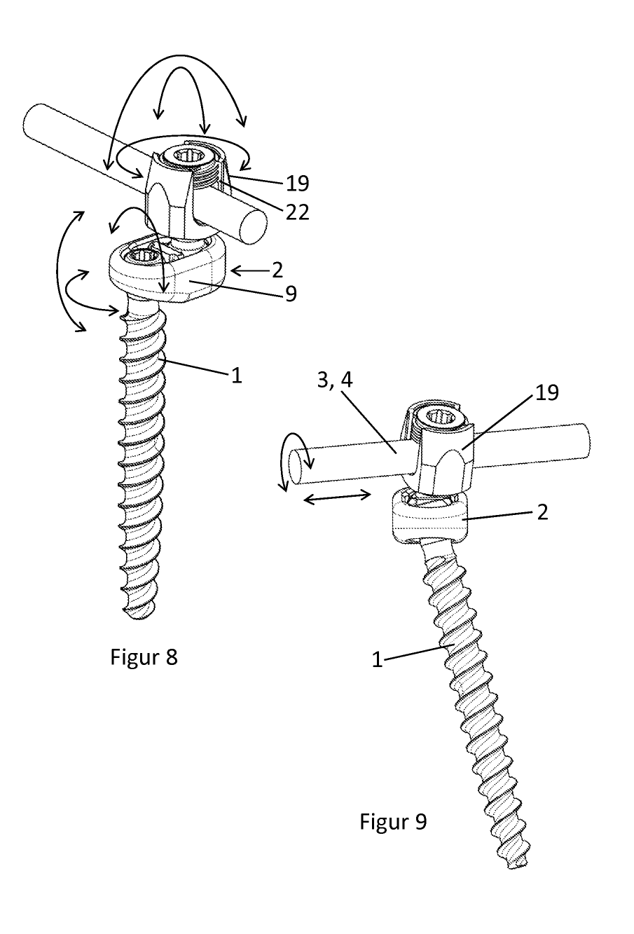

FIG. 7 shows a horizontal guide groove (34) in the connector element (9). This guide groove (34) receives the pins (31) of the clamping piece (28) with play and enables a horizontal, linear movement of the guided part of the clamping piece (28).

In the non-fixed or non-tensioned state, the pedicle screw (1) is engaged via its pedicle screw head (18) between the straps (29) of the clamping piece (28) and is situated in the first receptacle (10) of the connector element (9). In this state, the pedicle screw (1) is freely pivotable and rotatable. The spherical head (21) of the tulip (19) is engaged between the straps (30) of the clamping piece (28) and allows pivoting and rotation. A connecting rod (3,4) is inserted into the slot (22) of the tulip (19), and the locking element (25) is screwed in to such an extent that the connecting rod (3,4) can no longer be removed from the slot (22) in the longitudinal direction of the slot (22). The surgeon is now able to bring all the parts of a spinal fixation system (5) into the desired position which the surgeon requires for the spinal column to be operated on. Once this position is reached, it is sufficient merely to screw in the locking elements (25) of all the individual connectors (2) in order to fasten the entire fixation system (5) with a force fit.

By screwing in the locking element (25), this element presses on the connecting rod (3,4). The latter for its part displaces the pressure ram (26) against the housing bottom (15). The reaction force owing to the screwing-in force brings the spherical head (21) of the tulip (19) to bear on the right-hand U-shaped housing wall (14) of the connector element (9), whereby these parts are fixed. Since the contact of the spherical head (21) by the inner bead at the upper border of the housing wall (14) or the clamping surface (17) also produces a horizontal force component in the direction of the pedicle screw head (18), a slight displacement of the spherical head (21) horizontally in the direction of the pedicle screw (1) takes place, during which firstly the spherical head (21) comes to bear with a force fit on the clamping piece (28). The latter in turn comes to bear with a force fit on the pedicle screw head (18), which for its part comes to bear on a clamping surface (16) on the U-shaped housing wall (13) of the connector element (9). Thus, merely the screwing-in of the locking element (25) brings about a complete, force-fit connection between a connecting rod (3,4) and a pedicle screw (1). Loosening of the locking element (25) releases the entire fixing. FIGS. 8 and 9 illustrate the possible degrees of freedom of movement of the connector (2) according to the invention. Thus, the tulip (19) can be rotated by 360° about its centre axis in the connector element (9), while the connector (2) itself with the tulip (19) inserted is rotatable by 360° about the axial centre of the pedicle screw (1) (cf. FIG. 8). Likewise the connector element (9) with the tulip (19) inserted is tiltable about the spherical pedicle screw head (18), and the tulip (19) is tiltable about its spherical head (21), in each case in all directions (cf. FIG. 8).

In view of the fact that a connecting rod (3,4) can be displaced and rotated before fixing in the tulip (19) (cf. FIG. 9), the connector (2) according to the invention thus enables 8 degrees of freedom of movement in the non-fixed state.

A further configuration of the invention consists in that the pedicle screw (1) situated in the first receptacle (10) is polyaxially movably, but nondetachably connected to the connector element (9), so that the pedicle screw (1) can be screwed into a pedicle canal simultaneously with the connector element (9) and the polyaxial tulip (19), as a super-polyaxial pedicle so to speak.

### LIST OF REFERENCE NUMERALS

1 pedicle screw
2 connector
3,4 connecting rods
5 spinal fixation system
6 pedicles
7,8 connecting arrangements
9 connector element
10 1st receptacle
11 2nd receptacle
12 section of the housing bottom
13 U-shaped housing wall
14 U-shaped housing wall
15 housing bottom
16 clamping surface
17 clamping surface
18 pedicle screw head
19 tulip
20 tulip head
21 spherical head
22 slot
23 thread
24 bore
25 locking element
26 pressure ram
27 spring
28 clamping piece
29 strap
30 strap
31 pin
32 cylindrical surface
33 cylindrical surface
34 guide groove

1. Polyaxial connector for spinal fixation systems having a lateral connector element having two receptacles for polyaxial connecting arrangements,
the first receptacle, which is situated at one end of the connector element and is accessible from the upper and lower side of the latter, serving for connection to a spherical pedicle screw head and being formed substantially as a ball-and-socket joint bearing for receiving the pedicle screw head which is insertable from the lower side of the connector element,
and
the second receptacle, which is situated at the other end of the connector element, integrating a polyaxially movable tulip which projects to the upper side of the connector element and the upper part of which is provided, in a known manner, with a locking element for connection to a connecting rod of a spinal fixation system and for its own locking and the lower part of which, received in the connector element, has a spherical head having a pressure ram movably mounted therein and acted upon by a spring,
and
having a clamping piece which is mounted in a rotationally- and longitudinally-movable manner in the connector element between the receptacles and via which, with the aid of the locking element and the pressure ram, when fixing a connecting rod in a slot of the tulip, simultaneously a clamping force is exerted on the pedicle screw head situated in the first receptacle and via which an engagement of the pedicle screw head in the connector element is made possible by means of the spring.

2. Polyaxial connector according to claim 1, characterised in that the polyaxial tulip situated in the second receptacle has an internal thread and a locking element engaging therein and having an external thread such that, when screwing the locking element into the tulip, a connecting rod received in the slot of the tulip is firmly clamped and the pressure ram is simultaneously acted upon by a force directed in the direction of the inner housing bottom of the connector element.

3. Polyaxial connector according to claim 1, characterised in that the second receptacle of the connector element has an inner bead or a semi-annular enlargement as a clamping surface.

4. Polyaxial connector according to claims 1, characterised in that the clamping piece has in plan view a substantially H-shaped structure, of which the in each case two limbs on both sides as straps are provided with inner cylindrical surfaces in the longitudinal direction, in order on the one hand to be able to receive the spherical pedicle screw head and on the other hand to be able to receive the spherical head of the polyaxial tulip, and is made of resilient material.

5. Polyaxial connector according to claim 1, characterised in that the housing of the connector element has, in the region of the first receptacle, a section of the housing bottom, discernible in the longitudinal cross-section, which extends approximately from the centre towards the side obliquely upwards.

6. Polyaxial connector according to claim 1, characterised in that the housing of the connector element has only rounded corners towards the outside.

7. Polyaxial connector according to claim 1, characterised in that the locking, the securing of the non-tensioned pedicle screw against falling out and the releasing of the clamping mechanism, situated inside the connector element, are achieved with the aid of the spring situated on the pressure ram, the clamping piece mounted in a rotationally- and longitudinally-displaceable manner and a guide groove, provided in the first receptacle, for engaging the pedicle screw head of the pedicle screw, and the cylindrical surfaces of the clamping piece ensuring a tangible engagement.

8. Polyaxial connector according to claim 1, characterised in that a pedicle screw situated in the first receptacle is polyaxially movably, but nondetachably connected to the connector element, so that the polyaxial pedicle screw can be screwed into a pedicle canal simultaneously with the connector element and the polyaxial tulip.

9. Spinal fixation system comprising
pedicle screws having a spherical head,
polyaxial connectors according to claim 1
and
connecting rods.

10. Spinal fixation system comprising
polyaxial connectors according to claim 8 and
connecting rods.

11. Polyaxial connector (2) for spinal fixation systems having a lateral connector element (9) having two receptacles (10, 11) for polyaxial connecting arrangements (7,8),
the first receptacle (10), which is situated at one end of the connector element (9) and is accessible from the upper and lower side of the latter, serving for connection to a spherical pedicle screw head (18) and being formed as a ball-and-socket joint bearing for receiving the pedicle screw head (18) which is insertable from the lower side of the connector element (9),
and
the second receptacle (11), which is situated at the other end of the connector element (9), integrating a polyaxially movable tulip (19) which projects to the upper side of the connector element (9) and the upper part of which is provided with a locking element (25) for connection to a connecting rod (3,4) of a spinal fixation system (5) and for its own locking and the lower part of which, received in the connector element (9), has a spherical head (21) having a pressure ram (26) movably mounted therein and acted upon by a spring (27),
and
having a clamping piece (28) which is mounted in a rotationally- and longitudinally-movable manner in the connector element (9) between the receptacles (10, 11), a clamping force being exerted on the pedicle screw head (18) with the aid of the locking element (25) and the pressure ram (26) by means of a horizontal force component in the direction of the pedicle screw head (18) from the interaction of the spherical head (21) and a clamping surface (17) and is fixed in the connector element (9) by means of the horizontal force component.

12. Polyaxial connector (2) according to claim 11, characterised in that the polyaxial tulip (19) has an internal thread and a locking element (25) engaging therein and having an external thread such that, when screwing the locking element (25) into the tulip (19), a connecting rod (3,4) received in the slot (22) of the tulip (19) is firmly clamped and the pressure ram (26) is simultaneously acted upon by a force directed in the direction of the inner housing bottom (15) of the connector element (9).

13. Polyaxial connector (2) according to claim 11, characterised in that the second receptacle (11) of the connector element (9) has an inner bead or a semi-annular enlargement as a clamping surface (17).

14. Polyaxial connector (2) according to claim 11, characterised in that the clamping piece (28) has in plan view a substantially H-shaped structure, of which the in each case two limbs on both sides as straps (29, 30) are provided with inner cylindrical surfaces (32, 33) in the longitudinal direction, in order on the one hand to be able to receive the spherical pedicle screw head (18) and on the other hand to be able to receive the spherical head (21) of the polyaxial tulip (19), and is made of resilient material.

15. Polyaxial connector (2) according to claim 11, characterised in that the housing bottom (15) of the connector element (9) has, in the region of the first receptacle (10), a section (12) which extends obliquely upwards.

16. Polyaxial connector (2) according to claim 11, characterised in that the housing of the connector element (9) has only rounded corners towards the outside.

17. Polyaxial connector (2) according to claims 11, characterised in that the fixing, the securing of the non-tensioned pedicle screw (1) against falling out and the releasing of the clamping mechanism, situated inside the connector element (9), are achieved with the aid of the spring (27) situated on the pressure ram (26), the clamping piece (28) mounted in a rotationally- and longitudinally-displaceable manner and a guide groove (34), present in the first receptacle (10), for fixing the pedicle screw head (18) of the pedicle screw (1), and the cylindrical surfaces (32) of the clamping piece (28) ensuring a tangible fixing.
